(19) United States
(12) Patent Application Publication
(10) Pub. No.: US 2003/0100842 A1
(43) Pub. Date: May 29, 2003

(54) Method and kit for indicating the level of bad breath

(76) Inventors: Melvyn Nevo Rosenberg, Ramat-Gan (IL); Nir Sterer, Hod HaSharon (IL)

Correspondence Address: BAKER & BOTTS, 30 ROCKEFELLER PLAZA, NEW YORK, NY, 10112

(21) Appl. No.: 10/028,062

(22) Filed: Oct. 25, 2001

(51) Int. Cl. A61B 5/08

(52) U.S. Cl. 600/530

(57) ABSTRACT

The invention provides a method for the rapid assessment of the degree of halitosis comprising the steps of a) obtaining a sample of fluid and/or tissue from the oral cavity of a subject, b) assessing the amount of β-galactosidase in said sample, c) determining the degree of halitosis in said subject, by comparing the result obtained in step b) with appropriate reference values.

### FIELD OF THE INVENTION

This application is a continuation of International Publication Number WO 00/65033, published in English on Nov. 2, 2000, filed as International Application Number PCT/IL00/00240 on Apr. 24, 2000, which claims priority from U.S. Provisional Patent Application No. 60/130,970 filed on Apr. 26, 1999.

The present invention is concerned with a method for gauging the presence and degree of bad breath. More specifically, a method for the measurement of bad breath based on the estimation of β-galactosidase activity is disclosed, together with a diagnostic kit that employs this method.

### BACKGROUND OF THE INVENTION

Oral malodor, (halitosis, fetor ex ore) is a common human condition dating back to ancient times. Bad breath usually originates within the oral cavity itself, due to the production of putrid smelling gases by deposits of microorganisms, generally under anaerobic conditions.

Bad breath is considered to be caused chiefly by proteolytic activity of gram negative organisms. When gram positive bacteria from the mouth are incubated in the presence of amino acids, little or no odor ensues. However when gram negative bacteria are incubated in the presence of amino acids, putrid odors abound.

One of the practical problems related to this condition is that self-measurements of oral malodor are not reliable. This results, on the one hand, in patients who suffer from bad breath and are not aware of it, and on the other hand, in patients who are concerned about having this problem, while in fact they do not suffer from it. Because of the difficulties inherent in self-estimation of oral malodor, simple testing devices are of potential importance.

Various tests have been proposed for measuring parameters associated with bad breath and the degree of improvement following therapy. One approach which is commonly used is to measure the degree of volatile sulfides using electronic apparatuses (such as the Halimeter, Interscan Corp., Chatsworth Calif.), or visual means (such as by precipitation of lead acetate).

However, the first technique involves apparatus which is expensive to purchase and the second is time consuming. Another test which measures proteolysis, the BANA test, requires extensive work-up, including a period of heating at elevated temperature in a specialized instrument.

In a previous patent, U.S. Pat. No. 5,270,174, a technique for measuring microbial activity associated with bad breath is described. It includes swishing the oral cavity with sterile liquid, followed by introduction of the expectorate to a vessel containing an indicator of oxygen consumption. This test is messy and the time required for a color change may be long.

There are currently no quick and simple procedures which allow an individual to objectively test their own level of bad breath. In many social situations such a test could be invaluable to avoid embarrassment or to forewarn an individual of the need to take some remedial action to treat the condition. Such a test could also be of value in clinical situations where a fast objective test of bad breath could assist in evaluating periodontal disease as well as measure the effect of various treatments.

It is a purpose of this invention to provide a simple and convenient assay for the detection of oral malodor.

It is a further purpose of this invention to provide an oral malodor assay that is useful both for home use and for the evaluation of oral diseases and their response to treatment, by health care professionals.

Other objects and advantages of the invention will become apparent as the description proceeds.

### SUMMARY OF THE INVENTION

It has now been unexpectedly found that an assessment of the presence and degree of bad breath can be obtained by measuring the level of β-galactosidase enzymes in samples taken from the oral cavity. These enzymes are not known to contribute directly to bad breath odors, and therefore it is a matter of some surprise that their detection and measurement may be used as an indicator of the presence and severity of halitosis.

The present invention is primarily directed to a method for the rapid assessment of the degree of halitosis comprising the steps of:

a) obtaining a sample of fluid and/or tissue from the oral cavity of a subject.

b) assessing the amount of β-galactosidase present in said sample.

c) determining the degree of halitosis in said subject, by comparing the result obtained in step b) with appropriate reference values.

The term “appropriate reference values” refers to any quantitative or qualitative value assigned to the amount of β-galactosidase found in the sample, be it a set of discrete values or a calibration curve, such as a graphical plot of β-galactosidase levels for samples taken from a group of subjects versus the results of another measurement of bad breath, for example mean whole-mouth odor scores (based on odor-judge scoring on an arbitrary intensity scale). This term also refers to the use of the mathematical equation that describes said calibration curve. Finally, this term is also used, in the case of semi-quantitative β-galactosidase level results, to refer to comparison of the β-galactosidase score with a predetermined odor-intensity scale based on such semi-quantitative enzyme measurements.

While any suitable fluid or tissue (e.g. superficial mucosal cells taken by scraping the lingual or buccal mucosa, periodontal pocket exudate, and so on) may be used in order to carry out the method of the invention, saliva is the preferred sample type.

In another aspect, the invention provides a kit for the rapid assessment of halitosis comprising:

a) means for obtaining a fluid and/or tissue sample from the oral cavity;

b) a substrate of β-galactosidase that undergoes a change in color or other discernible property when broken down by said β-galactosidase, adsorbed onto a solid support medium;

c) a color or intensity chart or instructions for determining the level of halitosis from the change in color of the solid support medium.

Any suitable β-galactosidase substrate possessing the properties described hereinabove may be used in the manufacture of the kit of the invention. However, a preferred substrate is X-gal (5-bromo-4-chloro-3-indolyl-β-D-galactopyranoside).

In one preferred embodiment of the kit of the invention, the solid support medium further comprises a gratuitous inducer of β-galactosidase. While any such gratuitous inducer may be used, in a preferred embodiment of the kit of the invention, said inducer is IPTG (isopropyl β-D-thiogalactoside).

All the above and other characteristics and advantages of the invention will be further understood from the following illustrative and non-limitative examples of preferred embodiments thereof.

### DETAILED DESCRIPTION OF PREFERRED EMBODIMENTS

### EXAMPLE 1

### Beta-Galactosidase Activity as an Indicator of Bad Breath

Four subjects complaining of bad breath were tested for the odor levels (based on odor judge scoring on an increasing intensity scale of 0-5, and measurement of volatile sulfides using a sulfide monitor [Interscan Corp., Chatsworth, Calif. model 1170]). In addition, the level of beta-galactosidase activity was measured based on a colorimetric assay as follows: a sample of the back of the tongue was taken with a plastic spoon. The sample was removed from the spoon by washing twice with 0.2 mL water which were pooled into a single sample. X-gal (5-bromo 4 chloro 3 indoyl beta D galactopyranoside and IPTG (isopropyl thiogalactopyranoside) were added (0.05 mL of a 20 mg/mL solution and 0.05 mL of a 50 mg/mL solution, respectively) and the samples were incubated for one hour at 37 degrees Celsius in ELISA plates. The relative amount of enzymatic activity was recorded as OD at 650 nm.

The results of these measurements are presented in Table I.

TABLE I

| | | | Volatile |
|---|---|---|---|
| Subject no. | ELISA OD | Judge Score | Sulfides (ppb) |
| 1 | 0.15 | 2 (slight) | 20 |
| 2 | 0.27 | 2.5 (slight-moderate) | 20 |
| 3 | 0.30 | 2.5 (slight-moderate) | 30 |
| 4 | 0.52 | 3 (moderate) | 50 |

It is clear that the increasing ELISA reading of beta-galactosidase activity is in association with the increasing odor judge scores and volatile sulfide levels.

It was further found that β-galactosidase activity in saliva or other oral samples can be measured by simply wetting absorbent discs containing β-galactosidase activity detecting agents and incubating them at room temperature for short periods of time. By comparing the amount of color produced to color standards, a semi-quantitative estimate of the β-galactosidase activity in the oral sample can be determined.

### EXAMPLE 2

### Color Test Using Paper Discs

Five mm discs of absorbent paper were cut from sheets of Whatman chromatographic paper (Whatman Ltd., Maidstone, England). 100 mg of X-gal were dissolved in 5 mL of dimethylformamide. A second solution of 100 mg IPTG in 2 mL of water was prepared. The two solutions were combined. Twenty microliters of the combined solution were applied to each of the above paper discs. The discs were then dried for 24 hours before use and then affixed to a plastic backing.

To use the test, subjects collected a small amount of saliva and used it to thoroughly wet the above treated discs. The discs were allowed to stand at room temperature for 10 minutes. The color generated was scored using a standard color scale provided to the subjects. Breath odor scores from each subject were also measured using a sulfide monitor. A significant degree of correlation was found between the test scores.

### EXAMPLE 3

### Use of Paper Discs to Monitor Treatment with Mouthrinse

Absorbent paper discs impregnated with 20 mg/ml of X-gal and 50 mg/ml of IPTG were used in a self-administered test to evaluate breath odor before and after the use of a breath freshening mouthrinse with active ingredient compared to a placebo mouthrinse. The subjects saturated the discs with samples of their saliva immediately before and 1, 2 and 3 hours after using the treatment or placebo mouthrinse. The amount of color developed on the discs was scored after standing 5 minutes at room temperature. Breath odor scores were also measured with a sulfide monitor and an expert panel.

The results obtained with the three different measurements confirmed the breath freshening action of the treatment product compared to the control.

### EXAMPLE 4

### Paper Disc-Based Enzymatic Assay for the Assessment of Oral Malodor

The purpose of this study was to test a simple enzymatic color assay for the detection of oral malodor, to test its correlation with other oral malodor-related parameters such as odor judge scores and sulfide monitor measurements. In addition to the color assay, (organoleptic) measurements were made by two odor judges. Sulfide monitor measurements, microbial counts, BANA test and an indole test were similarly carried out.

1. Subjects

The study included 60 healthy young adult volunteers (mean age 23±2 years, 35 females). Subjects who were smokers or took antibiotics within one month prior to the study were not allowed to participate. The experiment was conducted according to an approved human subjects protocol and participants signed an informed consent form.

Participants were asked to refrain from eating or drinking for two hours prior to measurements. Initially, subjects were tested for malodor-related parameters: odor judge measurements, sulfide monitor levels, color assays and microbial counts. The subjects were split randomly into three groups: 1) treatment group: active chewing gum (with Breathanol TM). 2) placebo chewing gum (without Breathanol TM). 3) control group (no treatment). The subjects were given the chewing gum (or no treatment) and were asked to chew for 15 minutes. The subjects were reexamined after 1.5 and 3 hours following use. At the beginning of the experiment the subjects were asked to form an opinion on their own breath by scoring it using the same scale as the odor judges (see below).

2. Measurements Parameters:

Color Assay (OK2KS)

Paper discs (6 mm) were impregnated with enzyme substrates as described below:

The paper test was prepared by punching out 6 mm diameter discs from chromatography paper (Whatman paper no. 3). Two solutions were prepared, one by dissolving 100 mg of 5-bromo-4-chloro-3-indolyl-D galactopyranoside (X-Gal, Sigma) in 2 ml of N,N-Dimethylformamide (Sigma), and the other by dissolving 100 mg of isopropyl β-D-thiogalactoside (IPTG, Sigma) in 2 ml of double distilled water. 100 μl from each solution were combined, vortexed and then 20 μl of the mixture was impregnated on each paper disc. The discs were dried overnight at 37° C.

Saliva (whole, unstimulated) was collected from each subject at the beginning of the experiment (before treatment) as well as at 1.5 hours and after 3 hours. A 20 μL drop of each saliva sample was applied to the paper disc and following 10 min incubation at room temperature, the results were recorded after 10 minutes as follows: 0—no color, 1—faint color, 2—dark color.

3. BANA and Indole Production Assays

The BANA reagent card (PerioscanTM, Oral-B Laboratories, Redwood city, Calif.) and the indole production slide (DrySlideTM INDOLE, Difco laboratories, Detroit Mich.) were used according to manufacturer's instructions. Samples for these assays were taken from the same posterior tongue dorsum scrapings which were used to determine tongue odor scoring by the odor judges. Results were recorded as either: strong reaction=2, light reaction=1, or no color change=0.

4. Sulfide Monitor

Determination of intraoral headspace volatile sulfur compounds (VSC) was carried out using a sulfide monitor (model 1170, Interscan). Subjects were asked to refrain from talking for 5 minutes prior to measurements. The monitor was zeroed on ambient air, and the measurements were performed by inserting a disposable one quarter inch plastic straw approximately 4 cm into the partially opened oral cavity. Subjects were asked to breathe through their nose during measurements. Results were recorded as peak ppb sulfide equivalents.

5. Organoleptic Measurements

Two odor judges scored whole mouth malodor and for tongue malodor. For judge scoring of whole mouth subjects were instructed to exhale briefly through the mouth, at a distance of approximately 10 cm from the nose of the judge. Tongue malodor was scored by using a plastic spoon to scrape and scoop material from the far back region of the tongue dorsum, and scoring the malodor from the spoon by both judges, sequentially. Judge scores were recorded using a semi-integer scale of 0 to 5, as follows: 0, no appreciable odor; 1, barely noticeable odor; 2, slight, but clearly noticeable odor; 3, moderate odor; 4, strong odor; 5, extremely foul odor.

6. Microbial Counts

Viable counts from saliva samples were conducted using Diaslides (Savyon Diagnostics, Ashdod, Israel) containing tryptic soy agar (TSA) and mitis salivarius agar (MSA). Diaslides were incubated anaerobically for 72 hours at 37° C. Viable counts included total on TSA and MSA as well as counts of the blue colonies which formed on the MSA.

7. Statistical Analysis

Spearman correlation coefficients were used to determine the level of association between the various parameters. One way analysis of variance (ANOVA) was used to compare the results of the color assay (0, 1 and 2) in terms of the other parameters. Stepwise multiple regression analysis was carried out in order to test the contribution of the color test results and the sulfide monitor in predicting the odor judges scores.

8. Results

Spearman correlation coefficients comparing color assay scores for the three rounds of measurements (time 0, 1.5 and 3 hours) with the other parameters are presented in Table II. In this table, the appropriate p value is shown below each r value.

TABLE II

| | | Judge 1 | | Judge 2 | | | | | | |
|---|---|---|---|---|---|---|---|---|---|---|
| | Log | Whole | | Whole | | Microbial counts | | | Microbial assays | |
| | Monitor | mouth | Tongue | mouth | Tongue | TSA | MSA | Blue | BANA | Indole |
| Time zero | | | | | | | | | | |
| OK2KS | 0.18 | 0.39 | 0.50 | 0.47 | 0.48 | 0.29 | 0.37 | 0.38 | 0.21 | 0.21 |
| p = | 0.086 | 0.001 | <0.0001 | <0.0001 | <0.0001 | 0.013 | 0.002 | 0.002 | 0.055 | 0.057 |
| 1.5 Hours | | | | | | | | | | |
| OK2KS | 0.3290 | 0.32 | 0.42 | 0.46 | 0.33 | 0.31 | 0.42 | 0.39 | −0.12 | −0.12 |
| p = | 0.005 | 0.00 | <0.0001 | <0.0001 | 0.005 | 0.007 | <0.0001 | 0.002 | 0.185 | 0.171 |
| 3 Hours | | | | | | | | | | |
| OK2KS | 0.41 | 0.32 | 0.44 | 0.49 | 0.60 | 0.21 | 0.25 | 0.17 | 0.08 | 0.15 |
| p = | 0.001 | 0.006 | <0.0001 | <0.0001 | <0.0001 | 0.053 | 0.025 | 0.136 | 0.263 | 0.120 |

Among the various tests, OK2KS scores were most highly associated with the odor judge scores for whole mouth (p<0.007) and tongue (p<0.005) odor. Significant correlations were also observed between OK2KS and monitor measurements for the last two time points (p<0.005) and the microbial counts for the first two time points (p<0.013). In contrast, no significant association was found between OK2KS and the BANA or Indole production assays (p>0.055).

The initial correlations between OK2KS scores, sulfide monitor levels, BANA test results and microbial counts (MSA) are compared with odor judge scores in Table III below. Correlations between odor judge scores and OK2KS scores were as high or higher than corresponding correlations with sulfide monitor scores in all cases. Correlations between organoleptic scores and the BANA test were less significant, as were correlations between bacterial counts on MSA and odor judge scores. Indole scores were not correlated significantly with odor judge scores (not shown).

TABLE III

| | OK2KS | Sulfide Monitor | BANA | Bacterial |
|---|---|---|---|---|
| | Color Test | (ppb equivalents) | Test | Counts |
| Judge 1 | | | | |
| Whole mouth | | | | |
| r = | 0.39 | 0.37 | 0.25 | 0.27 |
| P = | 0.002 | 0.002 | 0.048 | 0.030 |
| Tongue | | | | |
| r = | 0.50 | 0.26 | 0.26 | 0.20 |
| P | <0.001 | =0.036 | =0.036 | =0.118 |
| Judge 2 | | | | |
| Whole mouth | | | | |
| R = | 0.47 | 0.46 | 0.22 | 0.16 |
| P | <0.001 | <0.001 | =0.086 | =0.196 |
| Tongue | | | | |
| r = | 0.48 | 0.38 | 0.14 | 0.18 |
| P | <0.001 | =0.002 | =0.282 | 0.160 |

Stepwise multiple regression analysis of odor judges scores for whole mouth and tongue odor (at time 0), in terms of color assay scores and log monitor readings are shown in Table IV.

TABLE IV

| Dependent | | OK2KS | Sulfide level | |
|---|---|---|---|---|
| variable | Intercept | coefficient | coefficient | Multiple r_ |
| Judge 1: whole | −1.38 | 0.42 | 0.68 | 0.54 |
| mouth | p = 0.0375 | 0.0137 | 0.0005 | p < 0.0001 |
| Judge 1: tongue | −0.062 | 0.57 | 0.47 | 0.57 |
| | 0.9148 | 0.0002 | 0.0062 | p < 0.0001 |
| Judge 2: whole | −1.54 | 0.62 | 0.70 | 0.60 |
| mouth | 0.028 | 0.0007 | 0.0006 | p < 0.0001 |
| Judge 2: tongue | 0.81 | 0.50 | 0.40 | 0.47 |
| | 0.2239 | 0.0036 | 0.0362 | p = 0.0007 |

Both sulfide monitor readings and OK2KS scores factored significantly into the regression equation for both judges scores for whole mouth and tongue odors, yielding multiple r values ranging from 0.47 (judge 2, tongue, p=0.0007) to 0.60 (judge 2, whole mouth, p<0.0001).

The results presented hereinabove show that OK2KS was highly significantly correlated with odor judges scores for whole mouth and tongue odor, at all three time points during the study. Furthermore, correlations between OK2KS and organoleptic scores were as significant, or more significant than corresponding correlations between the sulfide monitor and organoleptic scores. When multiple regression analysis was carried out to try to account for odor judge scores in terms of OK2KS and sulfide levels, both parameters entered into the regression equations, yielding multiple r values of up to 0.6. The results suggest that (I) OK2KS may be used as an assay which correlates with odor judge scores; and (ii) OK2KS can be used alongside sulfide monitor testing to improve the correlation with odor judge scores. The convenience and low anticipated cost of OK2KS may make it useful in both clinical and home settings.

While specific embodiments of the invention have been described for the purpose of illustration, it will be understood that the invention may be carried out in practice by skilled persons with many modifications, variations and adaptations, without departing from its spirit or exceeding the scope of the claims.

We claim:

1. A method for the rapid assessment of the degree of halitosis comprising
a) obtaining a sample of fluid and/or tissue from the oral cavity of a subject,
b) assessing the amount of β-galactosidase present in said sample, and
c) determining the degree of halitosis in said subject, by comparing the result obtained in step b) with appropriate reference values.

2. A method according to claim 1, wherein the sample of fluid is saliva.

3. A kit for the rapid assessment of halitosis comprising
a) a means for obtaining a fluid and/or tissue sample from the oral cavity,
b) a substrate of β-galactosidase that undergoes a change in color or other discernible property when broken down by said β-galactosidase, said substrate adsorbed onto a solid support medium, and
c) a color or intensity chart or instructions for determining the level of halitosis from the change in color and/or change in intensity of the solid support medium.

4. A kit according to claim 3, wherein the β-galactosidase substrate is 5-bromo-4-chloro-3-indolyl-β-D-galactopyranoside (X-gal).

5. A kit according to claim 3, wherein the solid support medium further comprises a gratuitous inducer of β-galactosidase.

6. A kit according to claim 5, wherein the gratuitous inducer of β-galactosidase is isopropyl β-D-thiogalactoside (IPTG).
